(19) United States
(12) Patent Application Publication
(10) Pub. No.: US 2007/0178581 A1
(43) Pub. Date: Aug. 2, 2007

(54) Screening method and apparatus for identifying a reactive compound bonding with a target compound

(75) Inventors: Yusuke Osaka, Kyoto-shi (JP); Morimasa Hayashi, Kyoto-shi (JP)

Correspondence Address: KANESAKA BERNER AND PARTNERS LLP, 1700 DIAGONAL RD, SUITE 310, ALEXANDRIA, VA, 22314-2848, US

(73) Assignee: SHIMADZU CORPORATION, Kyoto (JP)

(21) Appl. No.: 11/607,025

(22) Filed: Dec. 1, 2006

(30) Foreign Application Priority Data
Jan. 27, 2006 (JP) 2006-019551

(51) Int. Cl. G01N 33/558; C12M 3/00

(52) U.S. Cl. 435/287.2; 436/514

(57) ABSTRACT

An screening method and apparatus for online identification of a reactive compound bonding with a target compound includes a first dimension analysis step whereby a sample including a target compound and a reactive compound are injected together into a sample injection port. The sample is led to a first dimension column using a first dimension mobile phase and is separated into components. An isolation step isolates the sample portion into a loop passage. A reactive compound concentration step includes leading the reaction products into a trap column that selectively traps the reactive compound while dissociating it from the reaction products isolated by the isolation step. A second dimension analysis step leads the sample trapped in the trap column to a second dimension column using a second dimension mobile phase.

### BACKGROUND OF THE INVENTION AND RELATED ART STATEMENT

The present invention relates to a screening method and a apparatus for identifying a reactive compound bonding with a target compound.

In screening the bonding activity of a substance that bonds with a protein, the following off-line experimental steps have been conducted.
(1) A protein and a reactive compound are mixed to screen the compound that bonds with the protein.
(2) Only the protein is recovered from the mixture.
(3) The reactive compound bonded with the recovered protein is dissociated from the protein.
(4) The protein and the reactive compound are separated in a column, and only the reactive compound is recovered.
(5) The reactive compound recovered is measured using a mass spectrometer.

In another method, a reactive compound is labeled with a radioactive isotope and allowed to bond and react with protein. The amount of radiation emitted from the protein is then measured to verify the bonding between the reactive compound and protein.

It is labor intensive to obtain the results of the analysis by conducting all of the steps (1)-(5) described above off-line in the conventional method. Moreover, manual work is prone to potential errors as well as inconsistent results attributable to varying skill levels of researchers. In addition, conducting multiple step tasks off-line requires a researcher to work on the project for a prolonged period of time.

Accordingly, a screening method that allows these multiple steps to be performed on-line is desirable.

Further objects and advantages of the invention will be apparent from the following description of the invention.

### SUMMARY OF THE INVENTION

One aspect of a screening method includes the following steps (A) to (D), performed in that sequence, to identify a reactive compound bonding with a target compound:

(A) a first dimension analysis step, comprising injecting a sample including a target compound and a reactive compound into a sample injection port, the sample being led to a first dimension column, using a first dimension mobile phase, and separated into components,

(B) an isolation step, comprising isolating into a loop passage the sample portion containing the reaction products resulting from a bonding between the target compound and the reactive compound,

(C) a reactive compound concentration step, used as a concentration mobile phase, comprising dissociating said reactive compound from said reaction products using an eluant, leading the reaction products into a trap column thereby selectively trapping said reactive compound while dissociating it from the reaction products isolated by said isolation step, and

(D) a second dimension analysis step, comprising leading the sample trapped in said trap column to a second dimension column using a second dimension mobile phase for analysis.

Once a reaction solution consisting of a target compound and a reactive compound is prepared and injected into a sample injection port, the multiple steps (A) to (D) are performed on-line.

The present invention screens a reactive compound that bonds with a target compound to form reaction products, the “bond” therein includes not only chemical bonds, such as covalent, ionic, coordinate, and hydrogen bonds, but also physical bonds achieved by enclosure (a compound filling the void within a protein) or van der Waals force.

The “dissociation” of a reactive compound from reaction products means the undoing of a chemical or physical bond.

There are two methods for injecting a sample into the sample injection port: injecting a reaction solution, wherein target and reactive compounds are reacted beforehand, and sequentially injecting a target compound by itself and a reactive compound by itself.

Generally speaking, substances bonding with proteins, particularly when bonding through interactions occurring in vivo, are in an equilibrium state, wherein bonding and dissociation are repeated at a rapid pace. For this reason, preparing and injecting a sample into a sample injection port of a liquid chromatograph in the condition wherein a reactive compound is bonded with a target protein, may allow the mobile phase, used to move the sample to the first dimension column, to dilute the sample, thereby destroying the equilibrium state. Depending on the mobile phase, the sample that was in an equilibrium state might be dissociated and occasionally may become incapable of bonding again.

For example, in the case wherein the reactive compound is p-nitrophenyl-di-N-acetyl-β-chitobioside and the protein is WGA (wheat germ lectin), these substances apparently bond with one another through interactions. It has been found that when these substances are injected into a sample injection port of a liquid chromatograph in a reacted condition, p-nitrophenyl-di-N-acetyl-β-chitobioside and WGA are detected in the dissociated state. It has, therefore, been found difficult to accurately analyze reactive compounds bonding with proteins when the reactive compounds are p-nitrophenyl-di-N-acetyl-β-chitobioside, and the like.

One aspect of the present invention's screening method adapted to be applicable to a situation wherein the reaction product resulting from the bonding between a target compound and a reactive compound can be diluted and dissociated by a mobile phase, the sample injected into the sample injection port comprising of a target compound, by itself, and a reactive compound, by itself, having a smaller molecular weight than the target compound. The reactive compound is injected into the sample injection port first, followed by the injection of the target compound.

Moreover, in the first dimension column, a size-excluding column, is selected to vary the elution rate based on the molecular weight so that the target compound elutes first. This causes the target compound injected after the reactive compound to flow out first, thereby allowing the target compound to react with the reactive compound in the first dimension column.

A “size-excluding column” means a column capable of separating the components that are larger than a determined molecular size (molecular weight) from the components that are smaller than that determined size. In general, components having large molecular weights elute at faster rates and components having small molecular weights elute at slower rates in a size-excluding column. A size-excluding column allows users to set a desired molecular weight, i.e., a molecular weight exclusion limit, to be used for separation. This enables the molecular weight of a certain protein to be used for a reference point, to separate, for example, the components having smaller molecular weights than the protein.

In one aspect, the size-excluding column has a molecular weight exclusion limit not exceeding the size of a target compound. When such a size-excluding column is used, the reaction products resulting from the bonding between the target compound and the reactive compounds having smaller molecular weights than the molecular weight exclusion limit elute earlier, as in the case of the target compound, while the reactive compounds not bonding with the target compound elute later than the target compound and the reaction products since their molecular weights are smaller than the molecular weight exclusion limit. Thus, the reactive compounds bonded with the target compound can be separated from the reactive compounds not bonding with the target compound.

In the reactive compound concentration step, when leading the isolated reaction products into the trap column by using a concentrating mobile phase, it is preferable to further mix in the concentration mobile phase.

Although proteins are described herein as one example, the selection of target compounds is non-limiting.

The screening apparatus of the present invention for identifying a reactive compound bonding with a target compound comprises a first dimension passage, which leads the sample injected at the sample injection port to a first dimension column to separate the sample into components. The apparatus further comprises a sample supply unit, which injects the sample into the sample injection port; an isolation passage, which isolates the portion of the components containing reaction products resulting from the bonding between the target and reactive compounds into a loop passage; a reactive compound concentration passage, which leads the reaction products isolated by said isolation passage to a trap column used to selectively trap said reactive compound while dissociating it from the reaction products with an eluant used as a concentration mobile phase to dissociate said reactive compound from the reaction products; and a second dimension passage, which leads the sample trapped in said trap column to a second dimension column for analysis using a second dimension mobile phase to identify the reactive compound bonding with the target compound.

In cases wherein the reaction products of the target and reactive compounds are not readily dissociated through dilution by the mobile phase, the sample supply unit is controlled to inject a reaction solution of the target and reactive compounds as a sample.

In cases wherein the reaction products of the target and reactive compounds are readily dissociated through dilution by the mobile phase, the sample supply unit may be controlled to separately inject a target compound, by itself, and a reactive compound, by itself, having a smaller molecular weight than the target compound; the reactive compound is injected first, followed by the injection of the target compound. The first dimension column used in this case is a size-excluding column selected to vary the elution rate based on the molecular weight so that the target compound elutes before the reactive compound. Accordingly, the target compound injected after the reactive compound flows out first to allow the target compound to react with the reactive compound within the first dimension column.

In addition to the passage used to move the isolated reaction products using a concentrating mobile phase, it is preferable for the reactive compound concentration passage to include another passage for further mixing in a concentrating mobile phase.

Since the present invention's screening method is adapted to perform the first dimension analysis step (A), the isolation step (B), the reactive compound concentration step (C), and the second dimension analysis step (D), in that order, the multiple screening operations can be performed on-line.

In the first dimension analysis step (A), since a reaction solution prepared by mixing target and reactive compounds beforehand can be injected as a sample, in cases where the reaction products resulting from the bonding between the target and reaction compounds are not dissociated even when diluted by a mobile phase, the sample injection operation is simplified.

In cases wherein the reaction products of the target and reactive compounds are readily dissociated through dilution by the mobile phase, injecting the reactive compound followed by injection of the target compound and using a size-excluding column for the first dimension column to allow the reaction between the target and reactive compounds to occur within the first dimension column may prevent the mobile phase from diluting and dissociating the reaction products. Because this process isolates the reaction products while preventing dissociation, analysis of the reactive compound bonding with the target compound may be performed accurately.

In one aspect, using proteins as target compounds in the present invention's screening method allows for identification of the reactive compounds bonding with proteins.

One aspect of the present invention's screening apparatus includes a sample supply unit that allows for the injection of a sample at the sample injection port, a first dimension passage, an isolation passage, a reactive compound concentration passage, and a second dimension passage, all being connected together to enable on-line performance of the multiple screening operations.

Utilizing the sample supply unit in such a way as to inject a reaction solution consisting of reaction products of the target and reaction compounds as a sample may simplify the sample injection mechanism.

Utilizing the sample supply unit in such a way as to separately inject a target compound, by itself, and a reactive compound, by itself, having a smaller molecular weight than the target compound, the reactive compound being injected before the target compound, may separate the reactive compound that reacted with the target compound from the reactive compound that did not, even in cases wherein the reaction products of the target and reactive compounds dissociate when diluted by the mobile phase.

The apparatus may be adapted to allow for further mixing of the concentration mobile phase. Leading the isolated reaction products to the trap column with the concentrating mobile phase may facilitate dilution and thus dissociation of the reactive compounds from the target compound.

### BRIEF DESCRIPTION OF THE DRAWINGS

FIG. 1 is a schematic diagram of the passages in one aspect of the screening apparatus.

FIGS. 2A and 2B are the passage diagrams for the first dimension analysis step of the two-dimensional liquid chromatograph in according to the screening apparatus of FIG. 1, wherein FIG. 2A is the case wherein the reaction products containing protein is isolated, and FIG. 2B is the case wherein the unnecessary components are discharged.

FIG. 3 is a passage diagram showing one embodiment of the reactive compound concentration step according to the apparatus of FIG. 1.

FIG. 4 is a diagram showing the passages in one embodiment of the second dimension analysis step according to the apparatus of FIG. 1.

FIG. 5A is a schematic diagram of the passages used in another aspect of the screening apparatus according to FIG. 1.

FIG. 5B is a sample supply unit according to the screening apparatus of FIG. 1.

FIGS. 6A-6C illustrate the movement of the sample in the size-excluding column according to the screening apparatus of FIG. 1.

FIG. 7 is a waveform illustration showing one example of the chromatograms obtained from the eluate in the size-excluding column according to the screening apparatus of FIG. 1.

FIGS. 8A and 8B are charts showing the results of the analysis of the components eluted from the first dimension column of the screening apparatus according to FIG. 1, wherein FIG. 8A is the case wherein the target and reactive compounds are mixed beforehand and injected as a reaction solution to the sample injection port, and FIG. 8B is the case wherein the target and reactive compounds in the same example are injected separately into the sample injection port.

### DETAILED DESCRIPTION OF THE EMBODIMENTS

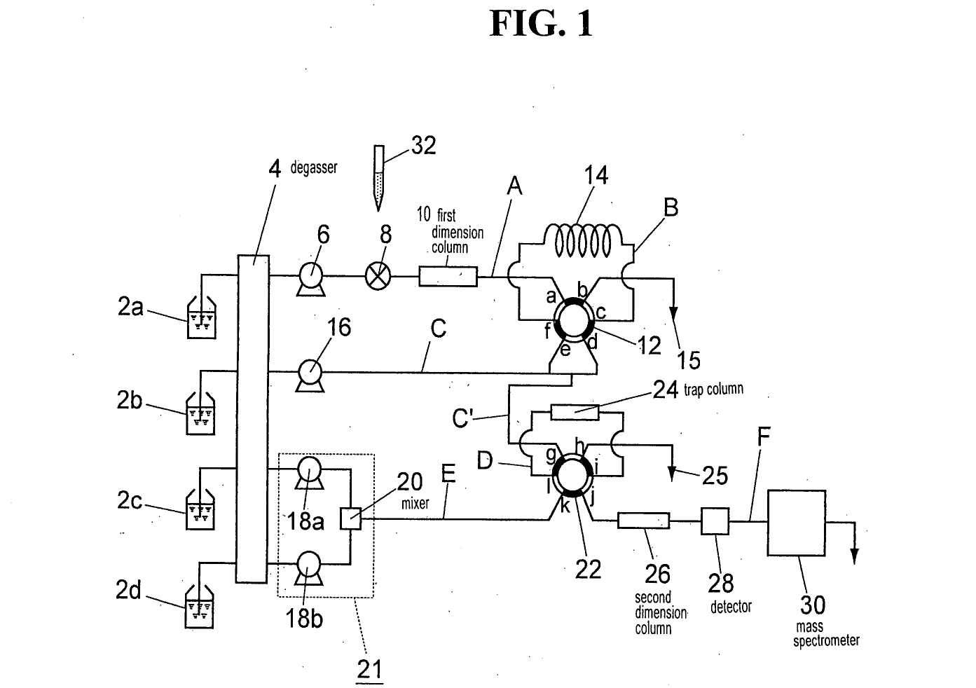

Examples of the present invention's screening method and the two-dimensional liquid chromatograph for screening used in the examples will be explained with reference to the drawings below. FIG. 1 is a schematic view of the passages used in one example of the present invention's two-dimensional liquid chromatograph. In this example, protein is used as the target compound, and the reactive compounds bonding with the protein are identified by screening.

For the purposes of this disclosure, a mixture of protein and several reactive compounds are described herein. Accordingly, the sample contains, in addition to the reaction products resulting from the bonding between the protein and certain reactive compounds, the compounds that did not bond with the protein, and could also contain unreacted protein.

One aspect of the screening apparatus disclosed herein includes a two-dimensional liquid chromatograph comprising a first dimension passage A for separating the sample into protein (including the reaction products of the protein and the bonded reactive compounds) and unreacted reactive compounds; an isolation passage B for isolating the reaction products from the separated sample components; a concentration mobile phase feeding passage C for feeding a concentration mobile phase; a reactive compound concentration passage D for leading the reaction products into a trap column 24 while dissociating the reactive compounds from the protein converted into the reaction products using the concentration mobile phase; a second dimension mobile phase feeding passage E for feeding a second dimension mobile phase used to move the reactive compounds trapped in the trap column 24; and a second dimension passage F for analyzing the reactive compounds moved by the second dimension mobile phase in a second dimension column 26. The screening apparatus also includes a sample supply unit that has a sample injection nozzle 32 for injecting a sample into a sample injection port 8 provided on the first dimension passage A. The passages A through F are connected to 6-port valves 12 and 22 capable of selectively connecting the passages.

The upstream end of the first dimension passage A is connected to a tank 2a, which stores a first dimension mobile phase. On the first dimension passage A, a degasser 4, a feeding pump 6, a sample injection port 8, and a first dimension column 10 are provided from the upstream side. The degasser 4 is provided to remove air bubbles or the like from the first dimension mobile phase fed by the feeding pump 6. A sample is injected from the sample injection port 8 using the sample injection nozzle 32, and the injected sample is led to the first dimension column 10 by the first dimension mobile phase in order to be separated into components, using, for example, a phosphoric acid buffer solution containing 0.8% sodium chloride, similar to that used in the first dimension mobile phase.

An isolation loop passage 14 is formed on the isolation passage B to store the components that are led to the isolation passage B. The upstream end of the concentration mobile phase feeding passage C is connected to a tank 2b that stores a concentration mobile phase. The concentration mobile phase is fed by a feeding pump 16 via the degasser 4. 0.1% FTA (trifluoroacetic acid), for example, is used as the concentrating mobile phase.

A trap column 24 is provided on the reactive compound concentration passage D to trap only the reactive compounds. The trap column 24 does not trap huge molecules such as protein, but selectively traps only reactive compounds, using an internal reverse phase column.

The upstream end of the second dimension mobile phase feeding passage E is connected to tanks 2c and 2d. For example, the tank 2c contains pure water containing 0.1% TFA, and the tank 2d contains acetonitryl containing 0.1% TFA, which are fed by feeding pumps 18a and 18b into a mixer 20 at predetermined feeding rates to be mixed to a predetermined mixing ratio. The mixture is then fed as a second dimension mobile phase. These feeding pumps 18a and 18b and the mixer 20 comprise a gradient feeding mechanism 21.

The second dimension passage F includes a second dimension column 26, a detector 28, and a mass spectrometer 30 from the upstream side. The detector 28 is, for example, an ultraviolet detector or a differential refractometer.

At the passage F, the sample is separated into components in the second dimension column 26, detected by the detector 28, and each component is identified by the mass spectrometer 30.

The 6-port valve 12 has ports a through f, and the 6-port valve 22 has ports g through l. The downstream end of the first dimension passage A is connected to port a of the 6-port valve 12. The upstream end of the isolation passage B is connected between ports c and f of the 6-port valve 12. The concentration mobile phase feeding passage C is connected to both ports d and e of the 6-port valve 12 to allow for the feeding of the concentration solution to port e to mix the liquid from port d with the concentration mobile phase. The concentration mobile phase feeding passage C is connected via the mixing passage C′ to port g of the 6-port valve 22 so as to feed the mixture of the liquid from port d and the concentration mobile phase to port g. The reactive compound concentration passage D is connected between ports i and l of the 6-port valve 22. The downstream end of the second dimension mobile phase feeding passage E is connected to port k of the 6-port valve 22. The upstream end of the second dimension mobile phase passage F is connected to port j of the 6-port valve 22. Port b of the 6-port valve 12 and port h of the 6-port valve 22 open to drains 15 and 25, respectively.

The 6-port valve 12 is adapted to block communications between ports a and f, between b and c, and between d and e when ports a and b, c and d, and e and f are respectively connected; likewise, communications between ports a and b, between c and d, and between e and f are blocked when ports a and f, b and c, and d and e are respectively connected.

The 6-port valve 22 is adapted to block communications between ports g and l, between h and i, and between j and k when ports g and h, i and j, and k and 1 are respectively connected; likewise, communications between ports g and h, between i and j, and between k and l are blocked when ports g and l, h and i, and j and k are respectively connected.

When the 6-port valve 12 is switched so that ports a and f, b and c, and d and e are connected, the first dimension passage A is connected to the isolation passage B and the sample components that have passed through the first dimension column 10 are transferred to the loop passage 14.

When the 6-port valve 12 is switched so that ports a and b, c and d, and e and f are connected, the downstream end of the first dimension passage A is connected to the drain 15 to discharge the sample that has passed through the first dimension column 10.

By switching the 6-port valve 12 at a certain timing to achieve port a-f, b-c, and d-e connections in this manner, the protein containing the reaction products separated from among the sample components in the first dimension column 10 can be transferred and isolated in the loop passage 14, while discharging any remaining, unnecessary, components at the drain 15.

When the 6-port valves 12 and 22 are switched to achieve connections between port a-b, c-d, and e-f and port g-l, h-i, and j-k, respectively, and a concentration mobile phase is fed from the concentration mobile phase feeding passage C, one portion of the concentration mobile phase fed by the feeding pump 16 is led via ports e and f into the loop passage 14, and together with the reaction products containing protein isolated in the loop passage 14, is led through the mixing passage C′ via ports c and d, as well as ports g and l, into the trap column 24.

At this time, the portion of concentration mobile phase fed by the feeding pump 16 that was not led to port e is mixed with the mobile phase from port d at the upstream end of the mixing passage C′ to dilute the mobile phase containing the protein that includes the reaction products. The reaction products, after coming into contact with the concentration mobile phase, are dissociated into protein and reactive compounds. The trap column 24 traps only the reactive compounds, and the remaining components, including the protein, are discharged via ports i and h through the drain 15.

When the 6-port valve 22 is switched so as to make port connections between g-h, i-j, and k-l, and a second dimension mobile phase is fed to the second dimension mobile phase feeding passage E, the second dimension mobile phase is led via ports k and l of the 6-port valve 22 to the reactive compound concentration passage D. The second dimension mobile phase led to the reactive compound concentration passage D causes the reactive compounds trapped in the trap column 24 to elute and then be led via ports i and j of the 6-port valve 22 to the second dimension passage F.

At the second dimension passage F, the reactive compounds led by the second dimension mobile phase are separated in the second dimension column 26, are detected by the detector 28, and the components thereof are identified by the mass spectrometer 30.

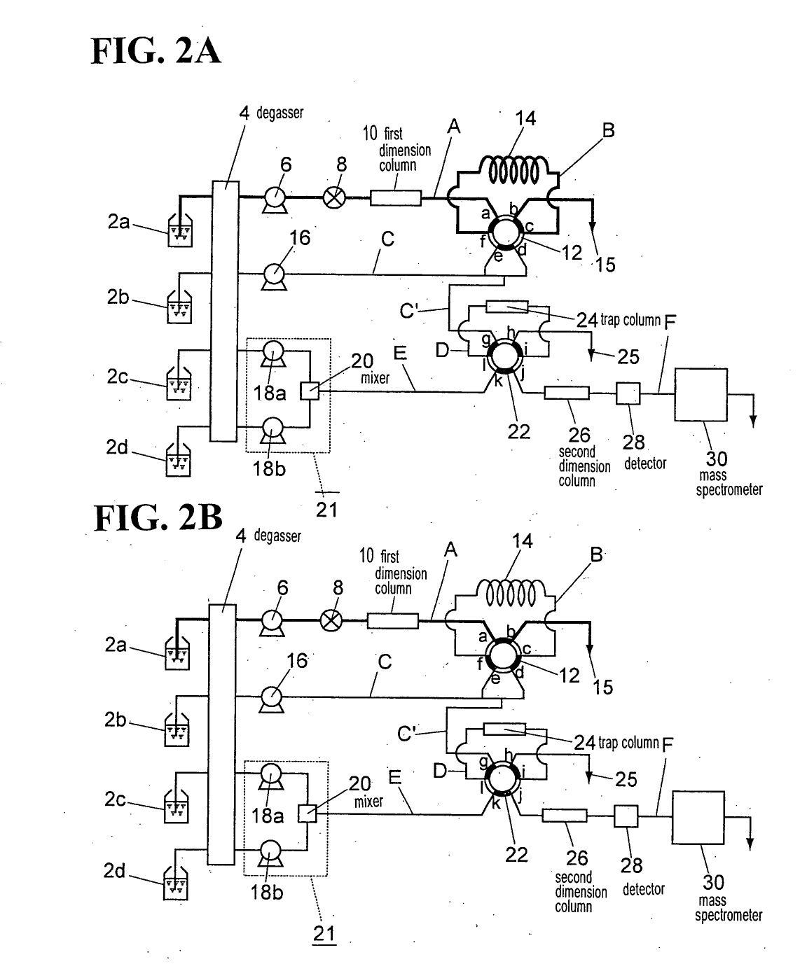

One example of the screening operations performed by using the two-dimensional liquid chromatograph described above will be explained below. The screening method described herein comprises the steps described below. FIGS. 2, 3, and 4 are schematic views of the passages used by the mobile phases during sample analysis, showing the first dimension analysis step, reactive compound concentration step, and second dimension analysis step, respectively.

### (First Dimension Analysis Step)

A sample, such as, a solution in which protein and reactive compounds are mixed together is used. The sample is prepared by a researcher beforehand.

As shown in FIG. 2, during the first dimension analysis step, a first dimension mobile phase is fed to the first dimension passage A. As shown in FIG. 2(A), once the sample is injected by the sample injection nozzle 32 into the sample injection port 8, the sample is transferred by the first dimension mobile phase to the first dimension column 10. The sample is then separated into a protein portion containing the reaction products generated by the reaction between the protein and certain reactive compounds, and an unreacted reactive compound portion. The protein portion containing the reaction products is transferred to the loop passage 14 when the 6-port valve 12 is switched to achieve ports connections a-f, b-c, and d-e. The sample components, other than the protein portion containing the reaction products, are as shown in FIG. 2(B), discharged through the drain 15 by switching the 6-port valve 12 to achieve port a-b, c-d, and e-f connections.

Because the flow rate of the first dimension mobile phase is constant, the time required for the protein portion containing the reaction products separated in the first dimension column 10 to reach the 6-port valve 12 can be calculated from the flow rate of the first dimension mobile phase and the distance between the first dimension column 10 and the 6-port valve 12. Based upon a time calculation, switching the 6-port valve 12 may allow only the protein portion containing the reaction products to be selectively transferred to the loop passage 14 and stored.

Although not shown in this example, a detector may be provided between the first dimension column 10 and the 6-port valve 12 so that the switching of the 6-port valve 12 is performed based on the signal transmitted by the detector.

### (Concentration)

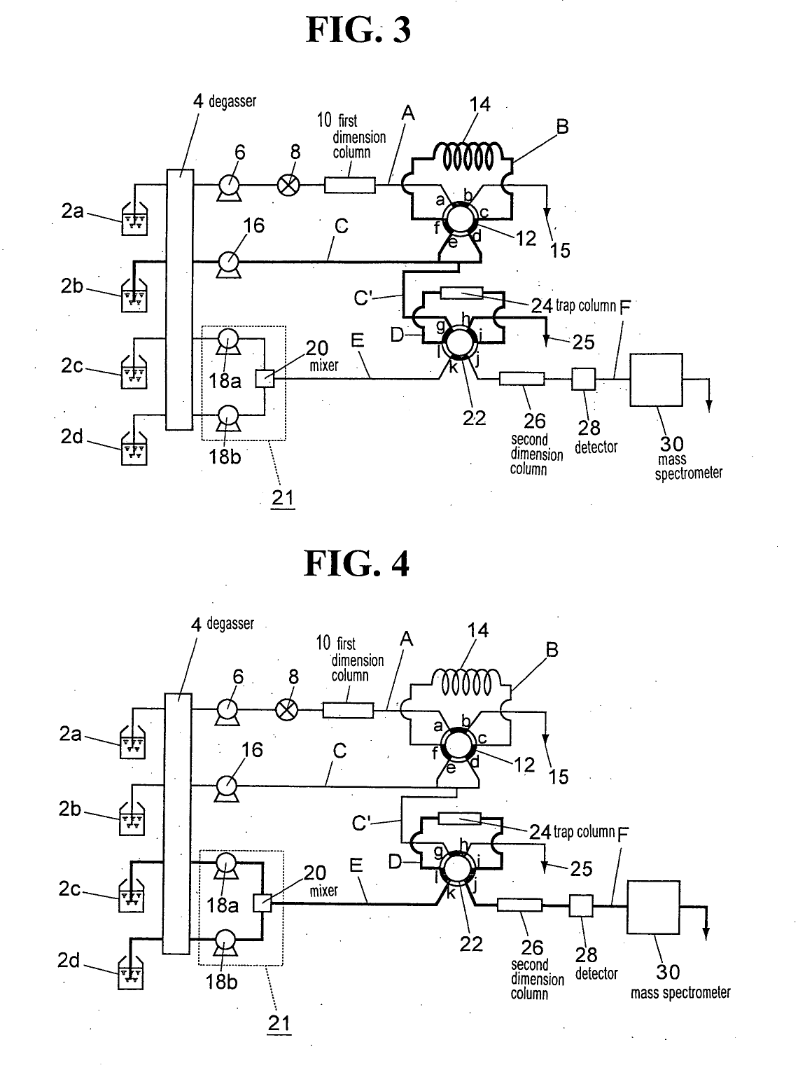

In the next step, a concentration mobile phase is fed to the passage indicated by a bold line in FIG. 3. The 6-port valve 12 is switched to make port a-b, c-d, and e-f connections, and the 6-port valve 22 is switched to make port g-l, h-i, and j-k connections. The concentration mobile phase is fed by the feeding pump 16 via ports e and f of the 6-port valve 12 to the loop passage 14. At this time, a portion of the concentration mobile phase flowing within the concentration mobile phase feeding passage C is not fed through port e, but is mixed with the solution from port d to flow into the mixing passage C′. The reaction products containing protein stored in the loop passage 14, together with the concentration mobile-phase, pass via port d through the mixing passage C′ to flow into port g of the 6-port valve 22. At this time, the reaction products come in contact with the concentration mobile phase, and the reactive compounds bonded with protein dissociate therefrom. The sample introduced to port g flows via port l into the concentration passage D, and only the reactive compounds are trapped by the trap column 24. The remaining components are discharged via ports i and h through the drain 25.

### (Second Dimension Analysis)

The 6-port valve 22 is switched to make port g-h, i-j, and k-l connections, and the gradient feeding mechanism 21 allows a second dimension mobile phase to flow through the passage, indicated by a bold line in FIG. 4, to be introduced via port k and l of the 6-port valve 22 to the trap column 24. The second dimension mobile phase causes the reactive compounds trapped in the trap column 24 to elute to then be introduced, together with the second dimension mobile phase, to the second dimension column 26 via port i and j of the 6-port valve 22 to be separated into components. Each component separated is detected by the detector 28 and is identified by the mass spectrometer 30.

According to the two-dimensional liquid chromatograph in this example, once a researcher prepares a sample by mixing -protein with reactive compounds, the steps of: separating the sample into reaction products comprising protein and unreacted reactive compounds; isolating the -reaction products containing protein; dissociating the reaction products; concentrating and trapping only the reactive compounds; and analyzing the reactive compounds can be performed on-line. Accordingly, the screening apparatus and methods described herein enable the researcher to perform these multiple screening operations on-line in a single analyzing system. Moreover, the on-line operations can be easily automated.

Automating the herein described operations, other than the sample preparation, enables an analysis in mass quantity under certain conditions, while preventing the occurrence of human error or obtaining inconsistent results attributable to different skill levels.

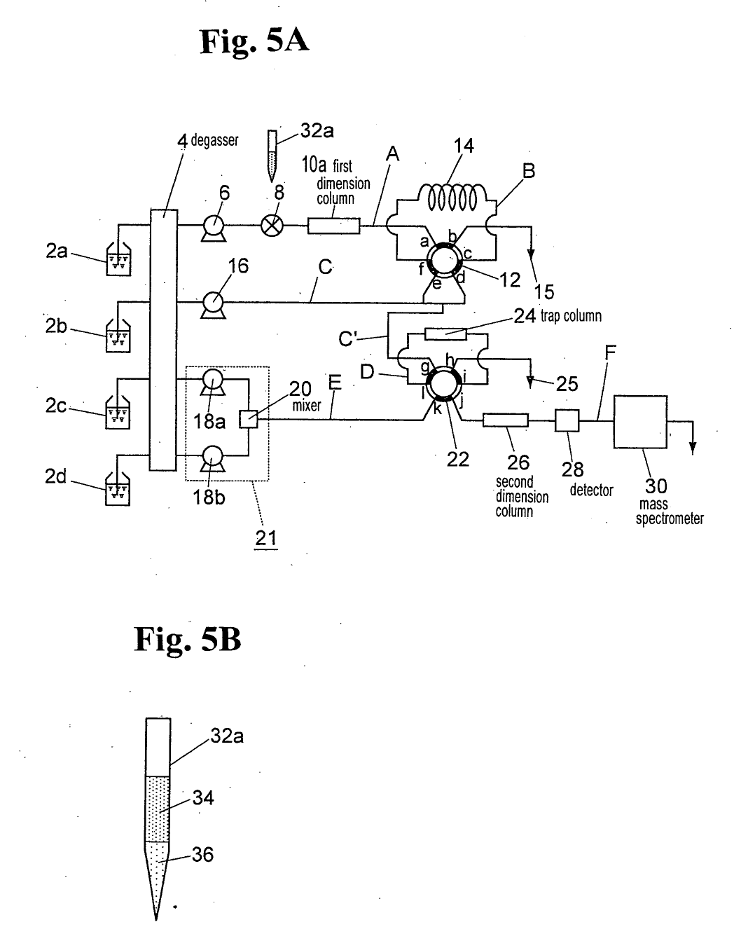

Depending on the combination of target compound, reactive compound, and the first dimension mobile phase, the above described method, wherein target and reactive compounds are mixed beforehand and injected to the sample injection port as a reaction solution, may occasionally allow the mobile phase to substantially dilute the reaction solution as they travel between the sample injection port 8 and the first dimension column 10, thereby dissociating the already bonded reactive compounds from the target compound. FIG. 5A illustrates such a case in which screening is performed according to the present invention's screening apparatus and method.

In FIG. 5A, the sample supply unit, which includes the sample injection nozzle 32a, is controlled to inject into the sample injection port 8, as a sample, a target compound or the protein itself and the reactive compounds having smaller molecular weights than that separately; the reactive compounds being injected before the target compound or protein.

Moreover, the first dimension column 10a is a size-excluding column selected to have varying elution rates based on the molecular weight so that the target compound or protein elute before the reactive compounds. Here, the size-excluding column serving as the first dimension column 10a is a gel permeation chromatography (GPC) column or a gel filtration chromatography (GFC) column. The molecular weight exclusion limit of the size-excluding column used in this example is of the same level as the molecular weight of the target protein compound and is capable of separating the protein having that or greater molecular weight from the reactive compounds having smaller molecular weights in order to be analyzed.

As shown in FIG. 5B, the sample injection nozzle 32a suctions the protein 34 first, and then suctions the reactive compounds 36. Thus, the layer of the reactive compounds 36 is formed at the tip, and the layer of the target compound 34 is formed at the base, within the sample injection nozzle 32a. When the sample is injected into the sample injection port 8, the reactive compounds 36 are injected first, followed by the target compound or protein 34. The sample injected in the sample injection port 8 is then moved to the first dimension column 10a in the order they were injected (i.e., the reactive compounds 36 first and then the protein 34). The analysis operations using the liquid chromatograph in this example are the same as in the first example, shown in FIGS. 2 through 4; the explanation is thus omitted.

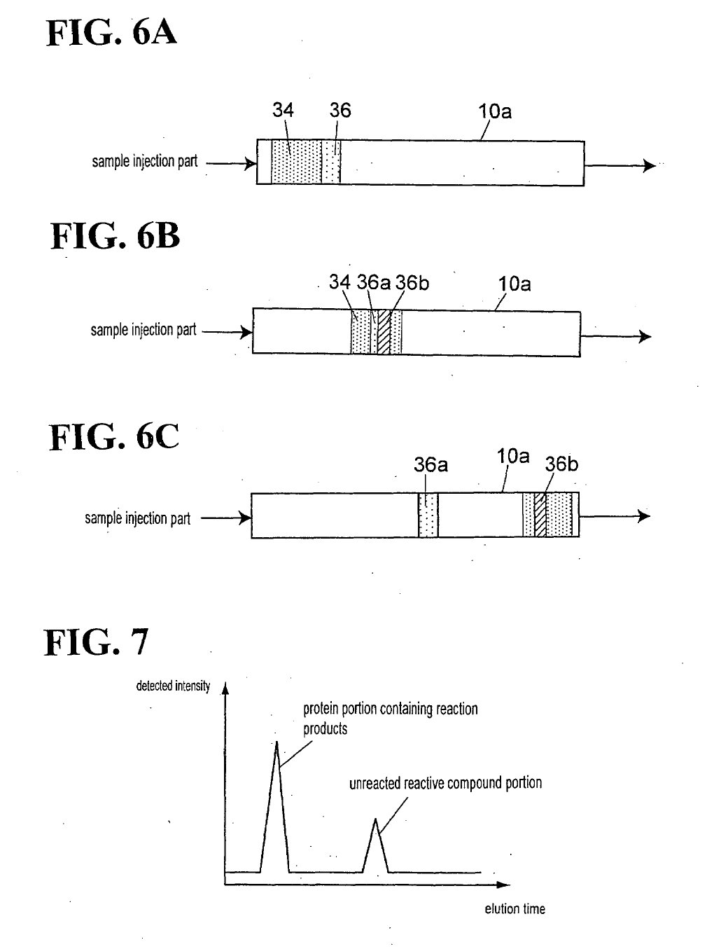

FIGS. 6A-6C show the movement of the sample in the first dimension column 10a. FIG. 6A shows a state immediately following the sample being introduced to the first dimension column 10a. In accordance with the order of injection, the reactive compounds 36 precede the protein 34. In the first dimension column 10a, however, the components having molecular weights that exceed the molecular weight exclusion limit (i.e., protein 34) move at a high speed, while the reactive compounds 36 having smaller molecular weights than the molecular weight exclusion limit move at a low speed.

Because of the difference in speed of travel, in the first dimension column 10a, the region of the protein 34 passes the region of the reactive compounds 36, as shown in FIG. 6B. At this time, the reactive compounds bonding with the protein 34 convert into reaction products 36b and move along with the protein 34 at a higher speed, whereas the reactive compounds not bonding with the protein 36a move at the same low speed. Eventually, as shown in FIG. 6C, the region of reaction products 36b bonded with protein elutes in the first dimension column 10a apart from the unreacted reactive compounds 36a.

As a result, a chromatogram consisting of the peak for the protein, which contains the reaction products 36b, and the peak for the unreacted reactive compounds, as shown in FIG. 7, can be obtained.

In this example, the 6-port valve 12 is switched to achieve port a-f, b-c, and d-e connections at the time the eluted protein portion containing the reaction products reaches the 6-port valve 12 (see FIG. 2A) to send and store the protein portion containing the reaction products in the loop passage 14. The unreacted reactive compound portion 36a, which eluted behind the protein portion containing the reaction products, is discharged to the drain 15 by switching the 6-port valve 12 to achieve port a-b, c-d, and e-f connections (see FIG. 2B).

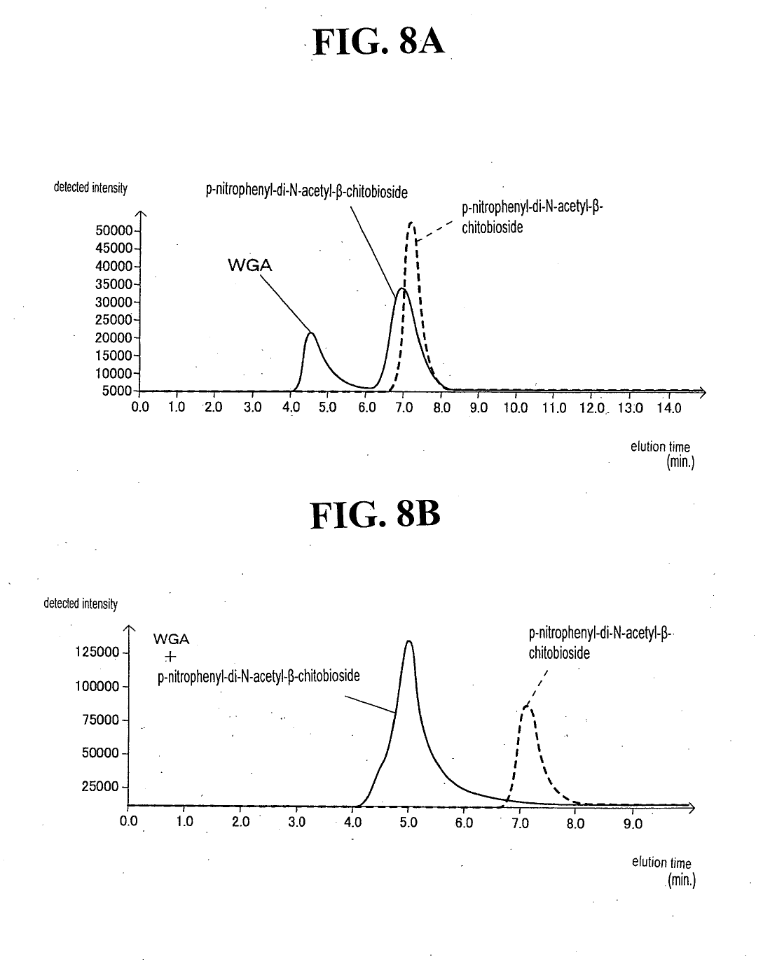

FIGS. 8A and 8B show the results of the analysis of the components eluted in the first dimension column in the example shown in FIG. 5. FIG. 8A shows the results in the case of injecting a reaction solution made by mixing the target compound and the reactive compounds beforehand into the sample injection port 8, and FIG. 8B shows the results in the case of injecting the target and reactive compounds separately in accordance with the same example.

The sample used consisted of WGA (1 mg/mL) for the target compound or protein, and p-nitrophenyl-di-N-acetyl-β-chitobioside (0.1 mg/mL) for the reactive compounds. For the first dimension mobile phase, 50 mM phosphoric acid buffer (pH 6.8)/0.8% NaCl solution was used, which was fed at the rate of 0.05 mL/minute. For the first dimension column 10a, a silica-based size-excluding DIOL column (product of Shimadzu Corporation) of 150 mm in length and 2 mm in internal diameter was used, while setting the column oven temperature at 25° C.

In the measurements that produced the chromatogram shown in FIG. 8B, 30 μL of WGA or protein was suctioned by the sample injection nozzle 32a, and then 5 μL of p-nitrophenyl-di-N-acetyl-β-chitobioside was suctioned, followed by the injection of the sample from the sample injection nozzle 32a into the sample injection port 8.

In FIGS. 8A and 8B, the peaks indicated by the broken lines are the peaks obtained when p-nitrophenyl-di-N-acetyl-β-chitobioside alone was injected into the sample injection port. The peaks of p-nitrophenyl-di-N-acetyl-β-chitobioside appear in different locations in FIGS. 8A and Bbecause the scale on the horizontal axis differs, but the retention time is identical in both graphs.

As shown in FIG. 8A, the elution time of p-nitrophenyl-di-N-acetyl-β-chitobioside, although affected by WGA, is not changed significantly compared to the case wherein p-nitrophenyl-di-N-acetyl-β-chitobioside alone was injected. This indicates that p-nitrophenyl-di-N-acetyl-β-chitobioside has been dissociated from WGA, or protein. In contrast, FIG. 8B illustrates, no peak appearing at the location of the elution time for p-nitrophenyl-di-N-acetyl-β-chitobioside alone, indicating that p-nitrophenyl-di-N-acetyl-β-chitobioside, as a result of reaction with WGA, is shifted to the location of the elution time for WGA.

These results show that, in the case of using p-nitrophenyl-di-N-acetyl-β-chitobioside and WGA as a sample, mixing them beforehand in order to prepare a reaction solution to be injected into the sample injection port 8, allows the mobile phase to dilute the sample and further allows p-nitrophenyl-di-N-acetyl-β-chitobioside to dissociate from protein, making the screening of the reaction products impossible. On the other hand, injecting p-nitrophenyl-di-N-acetyl-β-chitobioside first, followed by the injection of WGA or protein, enables the screening of p-nitrophenyl-di-N-acetyl-β-chitobioside, which reacts with WGA.

In the above examples, the mass spectrometer 30 is installed on the downstream side of the detector 28. The present invention, however, is not limited to this configuration, and may be configured with a probe for sample titration on the downstream side of the detector 28, for example, to separately trap each component in a container, such as a plate.

The disclosures of Japanese Patent Applications NO. 2005-023072 filed on Jan. 31, 2005, and No. 2006-019551 filed on Jan. 27, 2006 are incorporated by reference in their entireties.

While the invention has been explained with reference to the specific embodiments of the invention, the explanation is illustrative and the invention is limited only by the appended claims.

What is claimed is:

1. A screening method for identifying a reactive compound bonding with a target compound, comprising:
a first dimension sample analyzing step including injecting a sample comprising a target compound and a reactive compound into a sample injection port, leading the sample to a first dimension column, and separating the sample into components using a first dimension mobile phase;
an isolating reaction step for isolating products resulting from a bonding between the target compound and the reactive compound in a loop passage;
a reactive compound concentration step for leading an eluant into a trap column, said eluant dissociating said reactive compound from said reaction products as a concentration mobile phase, for selectively trapping said reactive compound while dissociating it from the reaction products isolated by the isolation of the sample; and
a second dimension analysis step for analyzing the sample trapped in said trap column while leading the sample to a second dimension column using a second dimension mobile phase for analysis.

2. The screening method according to claim 1, wherein the sample injected into said sample injection port is a reaction solution that the target and reactive compounds are reacted beforehand.

3. The screening method according to claim 1, wherein the sample injected into said sample injection port is a target compound, by itself, and a reactive compound, by itself, having a smaller molecular weight than the target compound;
wherein the injection into said sample injection port is performed in an order of the reactive compound first, followed by the target compound;
wherein said first dimension column is a size-excluding column selected to vary an elution rate based on a molecular weight so that said target compound elutes before said reactive compound;
whereby the target compound injected after the reactive compound is allowed to flow out of said first dimension column in order to allow the reaction between the target and reactive compounds to occur within said first dimension column.

4. The screening method according to claim 1, wherein said reactive compound concentration step includes mixing in said concentration mobile phase while leading the isolated reaction products to said trap column.

5. The screening method according to claim 1, wherein injecting the target compound comprises injecting a protein.

6. A screening apparatus configured to identify a reactive compound bonding with a target compound, comprising:
a first dimension passage configured to lead a sample injected into a sample injection port to a first dimension column using a first dimension mobile phase to separate the sample into components,
a sample supply unit configured to inject the sample into said sample injection port,
an isolation passage configured to isolate a portion of separated sample components containing reaction products resulting from bonding between the target and reactive compounds into a loop passage,
a reactive compound concentration passage for leading an eluant into a trap column, said eluant dissociating said reactive compound from said reaction products as a concentration mobile phase, for selectively trapping said reactive compound while dissociating it from the reaction products isolated by the isolation of the sample; and
a second dimension passage configured to lead the sample trapped in said trap column to a second dimension column using a second dimension mobile phase.

7. The screening apparatus according to claim 6, wherein said sample supply unit is configured to inject the sample comprising a reaction solution of the target compound and the reactive compound.

8. The screening apparatus according to claim 6, wherein said sample supply unit is configured to inject a target compound, by itself, and a reactive compound, by itself, the reactive compound comprising a smaller molecular weight than the target compound, said reactive compound being injected first and followed by said target compound; and
wherein said first dimension column is a size-excluding column configured to vary an elution rate based on a molecular weight so that said target compound elutes before said reactive compound, and the target compound injected after the reactive compound is allowed to flow out of said first dimension column before said reactive compound in order to allow a reaction between the target and reactive compounds to occur within said first dimension column.

9. The screening apparatus according to claim 6, wherein said reactive compound concentration passage includes a passage for transferring the reaction products isolated by the isolation by the concentration mobile phase, and another passage for further mixing said concentration mobile phase.

10. The screening apparatus according to claim 6, further comprising a two-dimensional liquid chromatograph.
